(19) United States
(12) Patent Application Publication
(10) Pub. No.: US 2017/0112743 A1
(43) Pub. Date: Apr. 27, 2017

(54) DECOLORIZATION OF DYED KERATIN FIBERS

(71) Applicant: Henkel AG & Co. KGaA, Duesseldorf (DE)

(72) Inventors: Juergen Schoepgens, Schwalmtal (DE); Burkhard Mueller, Duesseldorf (DE); Udo Erkens, Neuss-Grimlinghausen (DE)

(73) Assignee: Henkel AG & Co. KGaA, Duesseldorf (DE)

(21) Appl. No.: 15/399,690

(22) Filed: Jan. 5, 2017

Related U.S. Application Data
(63) Continuation of application No. PCT/EP2015/062391, filed on Jun. 3, 2015.

(30) Foreign Application Priority Data
Jul. 9, 2014 (DE) 10 2014 213 325.4

(51) Int. Cl. A61K 8/60; A61K 8/36; A61Q 5/08; A61K 8/41; A61K 8/49; A61K 8/34; A61K 8/19

(52) U.S. Cl. CPC A61K 8/602; A61K 8/342; A61K 8/36; A61K 8/19; A61K 8/411; A61K 8/4926; A61K 8/347; A61K 8/4973; A61Q 5/08

(57) ABSTRACT

Multi-component packaging unit (kit of parts) for the reductive decolorization of dyed fibers, which comprises separately formulated (I) a container (A) containing a cosmetic agent (a) and (II) a container (B) containing a cosmetic agent (b), wherein agent (a) in container (A) includes (a1) one or more reducing agents (a2) one or more fatty components, and has (a3) a water content of no more than 10.0% by weight, based on the total weight of agent (a), and agent (b) in container (B) has (b1) a water content of at least 30.0% by weight, based on the total weight of the agent (b). The invention further relates to a method for reductive decolorization of dyed keratin fibers using the multi-component packaging unit.

### FIELD OF THE INVENTION

The present invention generally relates to multicomponent packaging units (kit of parts) for the reductive decolorization of dyed keratinic fibers, which units contain separately formulated containers (A) and (B). In this case, container (A) includes a cosmetic agent (a), which is formulated substantially water-free and includes at least one selected reducing agent and one or more fatty components. Container (B) includes a cosmetic agent (b) which represents an aqueous formulation. A further subject of the present invention is a method for the reductive decolorization of dyed keratin fibers, said method in which the above-described multicomponent packaging unit is used.

### BACKGROUND OF THE INVENTION

Preparations for tinting and dyeing hair are an important type of cosmetic products. They may be used to lighten or darken the natural hair color according to the wishes of the particular person, to achieve a completely different hair color, or to cover unwanted color tones such as, for example, gray tones. Conventional hair dyes are formulated, depending on the desired color or permanence of the dyeing, either on the basis of oxidation dyes or on the basis of direct dyes. Combinations of oxidation dyes and direct dyes are often used to achieve special shades.

Dyes based on oxidation dyes lead to brilliant and permanent color tones. However, they require the use of strong oxidizing agents such as, for example, hydrogen peroxide solutions. Such dyes contain oxidation dye precursors, so-called developer components and coupler components. The developer components form the actual dyes under the influence of oxidizing agents or atmospheric oxygen with one another or during coupling with one or more coupler components.

Dyes based on direct dyes are often used for temporary dyeing. Direct dyes are dye molecules that are directly absorbed onto the hair and do not require any oxidative process to develop the color. Important representatives of this class of dyes are, for example, triphenylmethane dyes, azo dyes, anthraquinone dyes, or nitrobenzene dyes, each of which can carry cationic or anionic groups.

It can occur in all of these dyeing processes that for various reasons the color is to be made totally or partially reversible. A partial removal of the color is recommended, for example, if the coloring result on the fibers is darker than desired. On the other hand, a complete removal of the color can also be desired in some cases. Thus, it is conceivable, for example, that the hair is to be colored or tinted to a certain shade for a specific reason and after some days the original color is to be restored.

Agents and methods for removing color are already known from the literature. A method, quite well known from the prior art, to make colors reversible is the oxidative decolorization of dyed hair, for example, with the aid of a conventional bleaching agent. In this process, however, the fibers can be damaged by the use of strong oxidizing agents.

Further, reductive processes for removing color have already been described. Thus, for example, European patent application EP 1300136 A2 discloses a method for hair treatment in which the hair is colored in a first step and is again decolorized reductively in a second step. In so doing, the reductive decolorization occurs by using a formulation containing a dithionite salt and a surfactant. In WO 2008/055756 A2, the reductive decolorization of keratin fibers is carried out by means of a mixture of a reducing agent and an absorbent.

When reductive decolorizing agents are used, the decolorization occurs by the reduction of the dyes located on the keratin fibers or hair. The dyes are usually converted into the reduced leuco forms by the reduction. In this process, the double bonds in the dyes are reduced, the chromophore system of the dyes is disrupted in this manner, and the dye is converted into a colorless form.

To reduce the dyes, strong reducing agents are generally used, which can undergo unwanted reactions with oxidizing agents, atmospheric oxygen being one of these, for example. The reducing agents, moreover, are often not very stable in an aqueous solution and, depending on the pH of the solution, broken down more or less rapidly. For example, the reductive decolorizing agent, known from the prior art, sodium dithionite, is sensitive to atmospheric oxygen and breaks down slowly in an aqueous solution. These degradation reactions can be slowed down by increasing the pH. The establishing of a weakly alkaline pH stabilizes aqueous dithionite solutions, so that the solution can be stored for several weeks to months with the exclusion of oxygen. If the reductive decolorizing agent is to be stored for a longer time, however, or high temperatures predominate under delivery conditions, the formulation as a solution, in particular as a water-containing solution, is not the method of choice. To circumvent this problem, the reducing agents themselves are often used as a solid, for example, in powder form, in prior art documents. This approach is associated with various disadvantages, however.

The reducing agents must be dissolved in a cosmetic carrier before use. If they are incorporated in pure form into the cosmetic carrier for this purpose, for example, as a powder, dust can arise, which, if it were to be inhaled, can irritate the respiratory tract of the user. If rather large particle sizes of the powdered reducing agent are selected, there is the risk that the reducing agents do not dissolve sufficiently and thus lead to a nonuniform, unattractive decolorization result. In the case of incomplete dissolution, the full amount of the reducing agent is also not available for the decolorization process, so that the decolorization result in this case can turn out to be weaker than planned.

If the solubility of the reducing agent, formulated in the shape of particles, in the cosmetic carrier is poor, the user is forced, furthermore, to mix together the solid reducing agent and the cosmetic carrier for a very long period. This process is inconvenient for the user, time-consuming, and therefore greatly undesirable.

The object of the present invention, therefore, was to provide a decolorizing agent for the decolorization of dyed keratinic fibers, which decolorizes dyed keratin fibers as uniformly and effectively as possible. The decolorizing agent should be characterized by a high storage stability and still possess a high decolorizing power after long storage periods at high temperatures as well. The ready-to-use decolorizing agent should be easy to prepare for the user and convenient to apply. In particular, no dust should arise during use. Moreover, the consistency of the decolorizing agent should be optimized so that, on the one hand, it can be easily applied to the user's head and be readily distributed, but, on the other, not drip off the keratin fibers.

Surprisingly, it has now emerged that the aforementioned object can be achieved in an excellent way, if the reductive decolorizing agent is formulated as a multicomponent packaging unit (kit of parts), which comprises two separately formulated containers (A) and (B), wherein containers (A) and (B) each contain the cosmetic agents (a) and (b). In this case, agent (a) includes at least one reducing agent and one or more fatty components. Agent (a) is formulated as substantially free of water. Agent (b) is a water-containing cosmetic carrier. For the reductive decolorization of dyed keratinic fibers, the user combines agents (a) and (b) shortly before use and in this way prepares the ready-to-use decolorizing agent.

Furthermore, other desirable features and characteristics of the present invention will become apparent from the subsequent detailed description of the invention and the appended claims, taken in conjunction with this background of the invention.

### BRIEF SUMMARY OF THE INVENTION

A multicomponent packaging unit (kit of parts) for the reductive decolorization of dyed keratinic fibers, which comprises separately formulated from one another (I) a container (A) containing a cosmetic agent (a) and (II) a container (B) containing a cosmetic agent (b), wherein agent (a) in container (A) includes (a1) one or more reducing agent from the group comprising sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thio sulfate, hydroxymethanesulfinic acid, aminomethanesulfinic acid, cysteine, thiolactic acid, sulfanylacetic acid (thioglycolic acid), and/or ascorbic acid; and (a2) one or more fatty components from the group comprising C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, C₁₂-C₃₀ fatty acid esters, hydrocarbons, and/or silicone oils; and has (a3) a water content of at most 10.0% by weight, based on the total weight of agent (a); and agent (b) in container (B) has (b1) a water content of at most 30.0% by weight, based on the total weight of agent (b).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the present invention is a multicomponent packaging unit (kit of parts) for the reductive decolorization of dyed keratinic fibers, which comprises formulated separately from one another

(I) a container (A) containing a cosmetic agent (a) and
(II) a container (B) containing a cosmetic agent (b),
wherein
agent (a) in container (A) includes
(a1) one or more reducing agent from the group comprising sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, sulfanylacetic acid (thioglycolic acid), and/or ascorbic acid, and
(a2) one or more fatty components from the group comprising C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, C₁₂-C₃₀ fatty acid esters, hydrocarbons, and/or silicone oils,
   and has
(a3) a water content of at most 10.0% by weight, based on the total weight of agent (a), and
   agent (b) in container (B) has
(b1) a water content of at most 30.0% by weight, based on the total weight of agent (b).

Agents (a) and (b), which are included in containers (A) and (B) of the multicomponent packaging unit of the invention, are notable for an exceptionally good storage stability at high temperatures as well. Furthermore, it emerged during the work leading to this invention that the two agents (a) and (b) can be mixed with one another very conveniently and rapidly and that a very uniform decolorization result can be achieved with the ready-to-use decolorizing agent obtained after the mixing. In addition, no dust arises during the mixing of agents (a) and (b). It emerged, moreover, that hair damage could be reduced with the use of the decolorizing agent of the invention (i.e., during use of the mixture of agents (a) and (b)).

Keratinic fibers, keratin-containing fibers, or keratin fibers are to be understood to mean pelts, wool, feathers, and in particular human hair. Although the agents of the invention are primarily suitable for lightening and coloring keratin fibers or human hair, in principle nothing precludes use in other fields as well.

The term “dyed keratinic fibers” is understood to mean keratin fibers that were dyed with conventional cosmetic dyes known to the skilled artisan. In particular, “dyed keratinic fibers” are understood to mean fibers that were dyed with the oxidative dyes known from the prior art and/or with direct dyes. In this regard, reference is expressly made to known monographs, e.g., Kh. Schrader, Grundlagen and Rezepturen der Kosmetika [Fundamentals and Formulations of Cosmetics], 2^{nd} edition, Hüthig Buch Verlag, Heidelberg, 1989, which report the corresponding knowledge of the skilled artisan. Agents (a) and (b) contain the ingredients, essential to the invention, in each case in a cosmetic carrier, which in the case of agent (a) is substantially free of water and in the case of agent (b) includes water.

Agent (a) is formulated substantially water-free and can be present hereby in solid form, as a powder or as a paste. Preferably, agent (a) is formulated in the form of a paste. Agent (a) can comprise furthermore a solvent-containing carrier as well. Furthermore, the content of fatty components from group (a2) in agent (a) can also be selected to be so high that the fatty components function as a carrier of agent (a) and, therefore, apart from reducing agents (a1), represent the main component of agent (a).

Aqueous cosmetic agent (b) can be, for example, an agent with a suitable aqueous or aqueous-alcoholic carrier. For the purpose of reductive decolorization, such carriers can be, for example, creams, emulsions, gels, or surfactant-containing foaming solutions as well, such as, for example, shampoos, foam aerosols, foam formulations, or other preparations suitable for use on hair. The agents for reductive color removal from keratinic fibers are particularly preferably creams, emulsions, or flowable gels. Agent (b) is formulated particularly preferably as an emulsion.

Agent (a) in Container (A)

The multicomponent packaging unit (kit of parts) of the invention comprises a first separately formulated container (A) with a cosmetic agent (a). Agent (a) is characterized in that it includes as the first ingredient (a1), essential to the invention, at least one reducing agent from the group comprising sodium dithionite, zinc dithionite, potassium dithionite, sodium sulfite, sodium hydrogen sulfite, potassium sulfite, potassium hydrogen sulfite, ammonium sulfite, hydroxymethanesulfinic acid, aminomethanesulfinic acid, cysteine, thiolactic acid, sulfanylacetic acid (thioglycolic acid), and/or ascorbic acid.

Agent (a) includes as the second ingredient (a2), essential to the invention, one or more fatty components from the group comprising C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, C₁₂-C₃₀ fatty acid esters, hydrocarbons, and/or silicone oils.

Furthermore, it is characteristic and essential to the invention for agent (a) that it is present formulated substantially as water-free. The term “substantially water-free” in this case is understood to mean that the water content of agent (a) is at most 10.0% by weight. Preferably, the water content of agent (a), however, is below 10.0% by weight and is preferably at most 8.0% by weight, more preferably at most 5.0% by weight, even more preferably at most 3.0% by weight, and very particularly preferably at most 1.0% by weight. In this regard, all quantities given in percentage by weight refer to the total weight of agent (a).

Agent (a) includes as the first ingredient (a1), essential to the invention, at least one reducing agent from the group comprising sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, sulfanylacetic acid (thioglycolic acid), and/or ascorbic acid.

Sodium dithionite is an inorganic reducing agent with the molecular formula Na₂S₂O₄ and the CAS No. 7775-14-6.

Zinc dithionite is an inorganic reducing agent with the molecular formula ZnS₂O₄ and the CAS No. 7779-86-4.

Potassium dithionite is an inorganic reducing agent with the molecular formula K₂S₂O₄ and the CAS No. 14293-73-3.

Sodium sulfite is an inorganic reducing agent with the molecular formula Na₂SO₃ and the CAS No. 7757-83-7.

Sodium hydrogen sulfite is an inorganic reducing agent with the molecular formula NaHSO₃ and the CAS No. 7631-90-5. Sodium hydrogen sulfite is preferably used in the form of an aqueous solution.

Potassium sulfite is an inorganic reducing agent with the molecular formula K₂SO₃ and the CAS No. 10117-38-1.

Potassium hydrogen sulfite is an inorganic reducing agent with the molecular formula KHSO₃ and the CAS No. 7773-03-7. Potassium hydrogen sulfite is preferably used in the form of an aqueous solution.

Ammonium sulfite is an inorganic reducing agent with the molecular formula (NH₄)₂SO₃ and the CAS No. 10196-04-0.

Sodium thiosulfate is an inorganic reducing agent with the molecular formula Na₂S₂O₃ and the CAS No. 7772-98-7.

Potassium thiosulfate is an inorganic reducing agent with the molecular formula K₂S₂O₃ and the CAS No. 10294-66-3.

Ammonium thiosulfate is an inorganic reducing agent with the molecular formula (NH₄)₂S₂O₃ and the CAS No. 7783-18-8.

Hydroxymethanesulfinic acid is an organic reducing agent with the formula HO—CH₂—S(O)OH and the CAS No. 79-25-4. Alternatively, hydroxymethanesulfinic acid is also called formaldehydesulfoxyl acid. Both the use of hydroxymethanesulfinic acid itself and also the use of the physiologically acceptable salts of hydroxymethanesulfinic acid, for example, the sodium salt and/or the zinc salt, are according to the invention. The use of sodium formaldehyde sulfoxylate (sodium hydroxymethanesulfinate, the sodium salt of hydroxymethanesulfinic acid) and/or zinc formaldehyde sulfoxylate (zinc hydroxymethanesulfinate, the zinc salt of hydroxymethanesulfinic acid) is accordingly likewise according to the invention.

Aminomethanesulfinic acid is an organic reducing agent with the formula H₂N—CH₂—S(O)OH and the CAS No. 118201-33-5. Both the use of aminomethanesulfinic acid itself and also the use of the physiologically acceptable salts of aminomethanesulfinic acid, for example, the sodium salt and/or the zinc salt, are according to the invention. The use of sodium aminomethanesulfinate (the sodium salt of aminomethanesulfinic acid) and/or zinc aminomethanesulfinate (the zinc salt of aminomethanesulfinic acid) is likewise according to the invention for this reason.

Cysteine (2-amino-3-sulfanylpropionic acid) according to the invention is understood to be D-cysteine, L-cysteine, and/or a mixture of D- and L-cysteine.

Thiolactic acid (2-sulfanylpropionic acid) is understood to be D-thiolactic acid, L-thiolactic acid, and/or a mixture of D- and L-thiolactic acid. Both the use of thiolactic acid itself and also the use of thiolactic acid in the form of a physiologically acceptable salt thereof are according to the invention. A preferred salt of thiolactic acid is ammonium thiolactate. Ammonium thiolactate is the ammonium salt of thiolactic acid (i.e., the ammonium salt of 2-sulfanylpropionic acid) (formula XX).

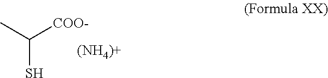

The definition of ammonium thiolactate comprises both the ammonium salts of D-thiolactic acid and the ammonium salts of L-thiolactic acid and mixtures thereof.

Sulfanylacetic acid (thioglycolic acid, 2-mercaptoacetic acid) is an organic reducing agent of the formula HS—CH₂—COOH; the compound has the CAS No. 68-11-1. In the case of thioglycolic acid, both the use of thioglycolic acid itself and the use of a physiologically acceptable salt of thioglycolic acid are according to the invention. For example, sodium thioglycolate, potassium thioglycolate, and/or ammonium thioglycolate can be used as physiologically acceptable salts of thioglycolic acid. Ammonium thioglycolate is a preferred physiologically acceptable salt of thioglycolic acid.

Ammonium thioglycolate is the ammonium salt of thioglycolic acid (i.e., the ammonium salt of sulfanylacetic acid) (formula XXX).

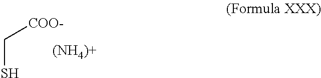

Ascorbic acid according to the invention is understood to be in particular (R)-5-[(S)-1,2-dihydroxyethyl]-3,4-dihydroxy-5H-furan-2-one (other alternative names: vitamin C, L-ascorbic acid) with the CAS No. 50-81-7.

The reducing agents from the group comprising sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, and/or ammonium thiosulfate have proven to be especially well suited for the reductive decolorization of oxidatively dyed hair. It is preferable for this reason, if agent (a) includes one or more reducing agent from this preferred group as reducing agent (a1).

Preferred, therefore, is a multicomponent packaging unit (kit of parts) for the reductive decolorization of dyed keratinic fibers, which is characterized in that
agent (a) in container (A) includes
(a1) one or more reducing agents from the group comprising sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, and/or ammonium thiosulfate.

Furthermore, the reducing agent(s) from group (a1) is/are preferably used in specific amount ranges. Preferably, agent (a) formulated substantially water-free includes the reducing agent(s) in a total amount of 10.0 to 90.0% by weight, preferably of 12.0 to 80.0% by weight, more preferably of 14.0 to 70.0% by weight, and particularly preferably of 16.0 to 60.0% by weight. These quantities given in percentage by weight hereby refer to the total weight of agent (a).

Particularly preferred, therefore, is a multicomponent packaging unit (kit of parts) for the reductive decolorization of dyed keratinic fibers, which is characterized in that
agent (a) in container (A) includes
(a1) one or more reducing agents from the group comprising sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, and/or ammonium thiosulfate in a total amount of 10.0 to 90.0% by weight, preferably of 12.0 to 80.0% by weight, more preferably of 14.0 to 70.0% by weight, and particularly preferably of 16.0 to 60.0% by weight, based on the total weight of agent (a).

Also particularly preferred is a multicomponent packaging unit (kit of parts) for the reductive decolorization of dyed keratinic fibers, which is characterized in that
agent (a) in container (A) includes
(a1) one or more reducing agents from the group comprising sodium dithionite, zinc dithionite, potassium dithionite, sodium thiosulfate, potassium thiosulfate, and/or ammonium thiosulfate in a total amount of 10.0 to 90.0% by weight, preferably of 12.0 to 80.0% by weight, more preferably of 14.0 to 70.0% by weight, and particularly preferably of 16.0 to 60.0% by weight, based on the total weight of agent (a).

It has emerged as especially advantageous, furthermore, if the agents of the invention contain specific combinations of reducing agents from group (a), because a very especially strong decolorizing effect is observed in specific combinations. Especially advantageous in this regard is the use of two different reducing agents from group (a1), wherein agent (a) includes
(a11) a first reducing agent, which is selected from the group comprising sodium dithionite, zinc dithionite, potassium dithionite, sodium thiosulfate, potassium thiosulfate, and/or ammonium thiosulfate, and in addition includes
(a12) a second reducing agent, which is selected from the group comprising sodium sulfite, sodium hydrogen sulfite, potassium sulfite, potassium hydrogen sulfite, and/or ammonium sulfite.

In other words, particularly preferred in the context of this embodiment is a multicomponent packaging unit for the reductive decolorization of dyed keratinic fibers, in particular human hair, wherein
agent (a) in container (A) includes
(a11) a first reducing agent, which is selected from the group comprising sodium dithionite, zinc dithionite, potassium dithionite, sodium thiosulfate, potassium thiosulfate, and/or ammonium thiosulfate, and in addition includes
(a12) a second reducing agent, which is selected from the group comprising sodium sulfite, sodium hydrogen sulfite, potassium sulfite, potassium hydrogen sulfite, and/or ammonium sulfite.

Agent (a) includes as the second ingredient (a2), essential to the invention, at least one or more fatty components from the group comprising C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, C₁₂-C₃₀ fatty acid esters, hydrocarbons, and/or silicone oils.

“Fatty components” in the context of the invention are understood to mean organic compounds with a solubility in water at room temperature (22° C.) and atmospheric pressure (760 mm Hg) of less than 1% by weight, preferably of less than 0.1% by weight.

The definition of fatty components includes explicitly only uncharged (i.e., nonionic) compounds. Fatty components have at least one saturated or unsaturated alkyl group having at least 12 C atoms. The molar weight of the fatty components is a maximum of 5000 g/mol, preferably a maximum of 2500 g/mol, and particularly preferably a maximum of 1000 g/mol. The fatty components are neither polyoxyalkylated nor polyglycerylated compounds. In this regard, polyalkoxylated compounds are compounds in the preparation of which at least 2 alkylene oxide units were reacted. By analogy, polyglycerated compounds are compounds in the preparation of which at least two glycerol units were reacted.

Because in the context of the present invention, explicitly only nonionic substances are regarded as fatty components, charged compounds such as, for example, fatty acids and salts thereof do not fall within the group of fatty components.

Preferred fatty components are the components from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, C₁₂-C₃₀ fatty acid esters, and hydrocarbons.

C₁₂-C₃₀ fatty alcohols can be saturated, mono- or polyunsaturated, linear or branched fatty alcohols having 12 to 30 C atoms.

Examples of preferred linear, saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol), and/or behenyl alcohol (docosan-1-ol).

Preferred linear, unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidonyl alcohol ((5Z,8Z,11Z,14Z)-eicosa-5, 8,11,14-tetraen-1-ol), erucyl alcohol ((13Z)-docos-13-en-1-ol), and/or brassidyl alcohol ((13E)-docosen-1-ol).

Preferred representatives of branched fatty alcohols are 2-octyldodecanol, 2hexyldodecanol, and/or 2-butyldodecanol.

A C₁₂-C₃₀ fatty acid triglyceride in the context of the present invention is understood to be the triester of the trihydric alcohol, glycerol, with three equivalents of fatty acids. In this regard, both structurally similar and also different fatty acids can be involved in ester formations within a triglyceride molecule.

Fatty acids according to the invention are understood to be saturated or unsaturated, unbranched or branched, unsubstituted or substituted C₁₂-C₃₀ carboxylic acids. Unsaturated fatty acids can be mono- or polyunsaturated. In the case of an unsaturated fatty acid, the C—C double bond(s) thereof can have the cis or trans configuration.

The fatty acid triglycerides are notable for particular suitability in which at least one of the ester groups proceeding from glycerol is formed with a fatty acid, which is selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

The fatty acid triglycerides can also be of natural origin. The fatty acid triglycerides, occurring in soybean oil, peanut oil, olive oil, sunflower oil, macadamia nut oil, Moringa oil, apricot kernel oil, Marula oil, and/or optionally hydrogenated castor oil, or mixtures thereof are especially suitable for use in agent (a).

A C₁₂-C₃₀ fatty acid monoglyceride is understood to be the monoester of the trihydric alcohol, glycerol, with a fatty acid equivalent. In this case, either the middle hydroxy group of glycerol or the terminal hydroxy group of glycerol can be esterified with the fatty acid.

Notable for particular suitability is the C₁₂-C₃₀ fatty acid monoglyceride in which a hydroxy group of glycerol is esterified with a fatty acid, wherein the fatty acids are selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], or nervonic acid [(15Z)-tetracos-15-enoic acid].

A C₁₂-C₃₀ fatty acid diglyceride is understood to be the diester of the trihydric alcohol, glycerol, with two fatty acid equivalents. In this case, either the middle and one terminal hydroxy group of glycerol can be esterified with two fatty acid equivalents, or however both terminal hydroxy groups of glycerol are each esterified with one fatty acid. Glycerol can be esterified hereby both with two structurally similar and with two different fatty acids.

Notable for particular suitability are fatty acid diglycerides in which at least one ester group proceeding from glycerol is formed with a fatty acid, which is selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

A C₁₂-C₃₀-fatty acid ester in the context of the present invention is understood to be the monoester of a fatty acid and an aliphatic, monohydric alcohol, the alcohol comprising up to 6 C atoms. Cited as suitable alcohols can be, for example, ethanol, n-propanol, isopropanol, 1-butanol, isobutanol, tert-butanol, n-pentanol, isopentanol, or n-hexanol. Preferred alcohols are ethanol and isopropanol.

Preferred C₁₂-C₃₀ fatty acid esters are the esters, formed during the esterification of the alcohols, ethanol and/or isopropanol, with one of the fatty acids from the group comprising dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9, 12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid. Of the fatty acid esters, isopropyl myristate is very particularly preferred.

Hydrocarbons are compounds consisting exclusively of carbon and hydrogen atoms and having 8 to 250 C atoms, preferably having 8 to 150 C atoms. Preferred in this regard are particularly aliphatic hydrocarbons such as, for example, mineral oils, liquid paraffin oils (e.g., liquid paraffin or light liquid paraffin), isoparaffin oils, semisolid paraffin oils, paraffin waxes, hard paraffin (solid paraffin), Vaseline, and polydecene.

Liquid paraffin oils (liquid paraffin and light liquid paraffin) have proven to be especially suitable in this regard. The hydrocarbon is very especially preferably liquid paraffin, also called white oil. Liquid paraffin is a mixture of purified, saturated, aliphatic hydrocarbons, which consists for the most part of hydrocarbon chains with a C-chain distribution of 25 to 35 C atoms.

Also preferred is a multicomponent packaging unit (kit of parts) for the reductive decolorization of dyed keratinic fibers, which is characterized in that
agent (a) in container (A) includes
(a2) one or more fatty components from the group comprising C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, C₁₂-C₃₀ fatty acid esters, and/or hydrocarbons.

Also preferred is a multicomponent packaging unit (kit of parts) for the reductive decolorization of dyed keratinic fibers, which is characterized in that
agent (a) in container (A) includes
(a2) one or more fatty components from the group, formed by dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol), (9Z)-octadec-9-en-1-ol (oleyl alcohol), (2E)-octadec-2-en-2-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidonyl alcohol ((5Z,8Z,11Z,14Z)-eicosa-5,8,11,14-tetraen-1-ol), erucyl alcohol ((13Z)-docos-13-en-1-ol), and/or brassidyl alcohol ((13E)-docosen-1-ol, 2-octyldodecanol, 2-hexyldodecanol, and/or 2-butyldodecanol.

Also preferred is a multicomponent packaging unit (kit of parts) for the reductive decolorization of dyed keratinic fibers, which is characterized in that
agent (a) in container (A) includes
(a2) one or more fatty components from the group of fatty acid triglycerides in which at least one of the ester groups proceeding from glycerol is formed with a fatty acid, which is selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

Also preferred is a multicomponent packaging unit (kit of parts) for the reductive decolorization of dyed keratinic fibers, which is characterized in that
agent (a) in container (A) includes
(a2) one or more fatty components from the group of hydrocarbons, formed by mineral oils, liquid paraffin oils, isoparaffin oils, semisolid paraffin oils, paraffin waxes, and/or hard paraffin (solid paraffin), Vaseline, and polydecenes.

The total content of fatty components (a2) in agent (a) has emerged as an essential adjustable variable in the reduction of dust formation. Dust formation is already reduced when smaller amounts of fatty components are used. To be able to ensure the most complete dust elimination possible, however, it has emerged as optimal to use fatty components (a2) in a total amount of at least 10% by weight. For this reason, it is particularly preferable, if agent (a) includes the fatty components (a2) in a total amount of 10.0 to 90.0% by weight, preferably of 20.0 to 86.0% by weight, more preferably of 25.0 to 84.0% by weight, and particularly preferably of 30.0 to 80.0% by weight, based on the total weight of agent (a). If fatty components (a2) were used in the indicated preferred and particularly preferred total amounts in agent (a), in addition, the reducing agent could be protected effectively from the effects of atmospheric oxygen and the storage stability could be significantly improved in this way.

Explicitly very particularly preferred, therefore, is a multicomponent packaging unit (kit of parts) for the reductive decolorization of dyed keratinic fibers, which is characterized in that agent (a) in container (A) includes the fatty component(s) from group (a2) in a total amount of 10.0 to 90.0% by weight, preferably of 20.0 to 86.0% by weight, more preferably of 25.0 to 84.0% by weight, and particularly preferably of 30.0 to 80.0% by weight, based on the total weight of agent (a).

The use of hydrocarbons has proven to be very particularly effective for reducing dust formation and for rendering the reducing agent inert to atmospheric oxygen. Paraffin oils and paraffin waxes, in particular, have proven to be very highly compatible with the solid inorganic reducing agents. For this reason, it is explicitly very particularly preferable to use as reducing agent (a2) one or more hydrocarbons in a total amount of 15.0 to 90.0% by weight, preferably of 20.0 to 85.0% by weight, more preferably of 25.0 to 80.0% by weight, and particularly preferably of 30.0 to 75.0% by weight, based on the total weight of agent (a).

Explicitly very particularly preferred, therefore, is a multicomponent packaging unit (kit of parts) for the reductive decolorization of dyed keratinic fibers, which is characterized in that
agent (a) in container (A) includes
(a2) one or more hydrocarbons in a total amount of 15.0 to 90.0% by weight, preferably of 20.0 to 85.0% by weight, more preferably of 25.0 to 80.0% by weight, and especially preferably of 30.0 to 75.0% by weight, based on the total weight of agent (a).

A further characterizing feature (a3), essential to the invention, of agent (a) is its water content of a maximum of 10% by weight, based on the total weight of agent (a).

Additional ingredients or active substances, which contain up to certain percentages of water, can be used in agent (a). It has emerged in this regard that a water content of up to 10% by weight does not detrimentally affect the storage stability of the agent (a) to a great extent.

To achieve an optimal storage stability at high temperatures as well, it has proven advantageous, however, if the water content of agent (a) is a value of at most 8.0% by weight, preferably at most 5.0% by weight, more preferably at most 3.0% by weight, and particularly preferably at most 1.0% by weight, based on the total weight of agent (a).

Also very particularly preferred, therefore, is a multicomponent packaging unit (kit of parts) for the reductive decolorization of dyed keratinic fibers, which is characterized in that
agent (a) in container (A) has
(a3) a water content of at most 8.0% by weight, preferably of at most 5.0% by weight, more preferably of at most 3.0% by weight, and particularly preferably of at most 1.0% by weight, based on the total weight of agent (a).

Agent (a) can also contain in addition further ingredients or active substances. In particular the use of nonionic surfactants (a4) in agent (a) has proven to be especially advantageous. It emerged that nonionic surfactants have a very good compatibility both with reducing agents (a1) and also in particular with fatty components (a2), so that agent (a) can be produced readily and reproducibly. An optimal miscibility with agent (b) could be achieved by the use of one or more nonionic surfactants.

The nonionic surfactant(s) can be used, for example, in total amounts of 0.1 to 15.0% by weight, preferably of 0.5 to 12.5% by weight, more preferably of 1.0 to 10.0% by weight, and especially preferably of 1.5 to 5.0% by weight, based on the total weight of agent (a).

Also preferred further therefore is a multicomponent packaging unit (kit of parts) for the reductive decolorization of dyed keratinic fibers, which is characterized in that
agent (a) in container (A), includes in addition
(a4) one or more nonionic surfactants in a total amount of 0.1 to 15.0% by weight, preferably of 0.5 to 12.5% by weight, more preferably of 1.0 to 10.0% by weight, and especially preferably of 1.5 to 5.0% by weight, based on the total weight of agent (a).

Surfactants are understood to be amphiphilic (bifunctional) compounds with at least one hydrophobic group and at least one hydrophilic molecular portion. The hydrophobic molecular portion is mostly a hydrocarbon chain having 10 to 30 carbon atoms. In the case of nonionic surfactants, the hydrophilic molecular portion comprises an uncharged, highly polar structural unit.

Nonionic surfactants contain as the hydrophilic group, e.g., a polyol group, a polyalkylene glycol ether group, or a combination of polyol and polyglycol ether groups. Such compounds are, for example,
adducts of 2 to 50 mol of ethylene oxide and/or 2 to 50 mol of propylene oxide to linear and branched fatty alcohols having 12 to 30 C atoms, fatty alcohol polyglycol ethers or fatty alcohol polypropylene glycol ethers or mixed fatty alcohol polyethers,
adducts of 2 to 50 mol of ethylene oxide and/or 2 to 50 mol of propylene oxide to linear and branched fatty acids having 6 to 30 C atoms, fatty acid polyglycol ethers or fatty acid polypropylene glycol ethers or mixed fatty acid polyethers,
adducts of 2 to 50 mol of ethylene oxide and/or 2 to 50 mol of propylene oxide to linear and branched alkylphenols having 8 to 15 C atoms in the alkyl group, alkyl phenol polyglycol ethers or alkyl polypropylene glycol ethers or mixed alkyl phenol polyethers,
adducts, end-capped with a methyl or C₂-C₆ alkyl group, of 2 to 50 mol of ethylene oxide and/or 2 to 50 mol of propylene oxide to linear and branched fatty alcohols having 8 to 30 C atoms, to fatty acids having 8 to 30 C atoms, and to alkylphenols having 8 to 15 C atoms in the alkyl group, such as, for example, the types obtainable under the marketing names Dehydol® LS and Dehydol® LT (Cognis),
C₁₂-C₃₀ fatty acid monoesters and diesters of adducts of 2 to 30 mol of ethylene oxide to glycerol,
adducts of 5 to 60 mol of ethylene oxide to castor oil and hydrogenated castor oil,
polyol fatty acid esters, such as, for example, the commercial product Hydagen® HSP (Cognis) or Sovermol® types (Cognis),
polyalkoxylated triglycerides,
polyalkoxylated fatty acid alkyl esters of the formula (Tnio-1)

R¹CO—(OCH₂CHR²)_{w}OR³ (Tnio-1)
in which R¹CO stands for a linear or branched, saturated and/or unsaturated acyl group having 6 to 22 carbon atoms, R² for hydrogen or methyl, R³ for linear or branched alkyl groups having 1 to 4 carbon atoms, and w for numbers from 2 to 20,
amine oxides,
hydroxy mixed ethers, as they are described, for example, in DE OS19738866,
sorbitan fatty acid esters and adducts of ethylene oxide to sorbitan fatty acid esters such as, for example, polysorbates,
sugar fatty acid esters and adducts of ethylene oxide to sugar fatty acid esters,
adducts of ethylene oxide to fatty acid alkanolamides and fatty amines,
sugar surfactants of the alkyl and alkenyl oligoglycoside type, or
sugar surfactants of the fatty acid-N-alkylpolyhydroxyalkylamide type.

C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and C₁₂-C₃₀ fatty acid esters do not have a highly polar end group (which is also evident in the low HLB values of the compounds in this group). In the context of the present invention, they are regarded as fatty components and according to the definition of the present invention therefore do not represent nonionic surfactants.

Furthermore, agents (a) can contain in addition one or more nonionic polymers (a5) as well.

The nonionic polymer(s) can be used, for example, in total amounts of 0.1 to 15.0% by weight, preferably of 0.2 to 10.5% by weight, more preferably of 0.25 to 7.5% by weight, and especially preferably of 0.3 to 5.0% by weight, based on the total weight of agent (a).

Also preferred further therefore is a multicomponent packaging unit (kit of parts) for the reductive decolorization of dyed keratinic fibers, which is characterized in that
agent (a) in container (A), includes in addition
(a5) one or more nonionic polymers in a total amount of 0.1 to 15.0% by weight, preferably of 0.2 to 10.5% by weight, more preferably of 0.25 to 7.5% by weight, and especially preferably of 0.3 to 5.0% by weight, based on the total weight of agent (a).

Polymers are understood to be macromolecules with a molecular weight of at least 1000 g/mol, preferably of at least 2500 g/mol, particularly preferably of at least 5000 g/mol, which consist of the same repeating organic units. Polymers are prepared by polymerization of a monomer type or by polymerization of various structurally different monomer types. If the polymer is prepared by the polymerization of one monomer type, the term homopolymers is used. If structurally different monomer types are used in the polymerization, the skilled artisan uses the term copolymers.

The maximum molecular weight of the polymer depends on the degree of polymerization (number of polymerized monomers) and is also determined by the polymerization method. In the context of the present invention, it is preferred if the maximum molecular weight of the zwitterionic polymer (d) is no more than 10⁷ g/mol, preferably no more than 10⁶ g/mol, and particularly preferably no more than 10⁵ g/mol.

Nonionic polymers are characterized in that they have no charges.

Examples of suitable nonionic polymers are vinylpyrrolidinone/vinyl acrylate copolymers, polyvinylpyrrolidinone, vinylpyrrolidinone/vinyl acetate copolymers, polyethylene glycol, ethylene/propylene/styrene copolymers, and/or butylene/ethylene/styrene copolymers.

Agent (b) in Container (B)

The multicomponent packaging unit of the invention comprises a second separately formulated container (B), which includes an agent (b). This agent (b) is a cosmetic carrier formulation which has a water content of at least 30.0% by weight, based on the total weight of agent (b).

In a preferred embodiment, agent (b) is formulated so that its water content has a value of at least 40.0% by weight, preferably of at least 50.0% by weight, more preferably of at least 55.0% by weight, and very particularly preferably of at least 60.0% by weight, based on the total weight of agent (b).

Also preferred further therefore is a multicomponent packaging unit (kit of parts) for the reductive decolorization of dyed keratinic fibers, which is characterized in that
agent (b) in container (B) has
(b1) a water content of at least 40.0% by weight, preferably of at least 50.0% by weight, more preferably of at least 55.0% by weight, and very particularly preferably of at least 60.0% by weight, based on the total weight of agent (b).

To optimize the decolorizing effect, the ready-to-use decolorizing agent, i.e., the mixture of agents (a) and (b), preferably has an acidic pH. Therefore, preparation (b) as well is adjusted preferably to an acidic pH of 1 to 6, preferably of 1.3 to 4.5, more preferably of 1.6 to 4.0, and particularly preferably of 2.0 to 3.6. The pH values were measured using a type N 61 glass electrode from the company Schott at a temperature of 22° C. To adjust the acidic pH, agent (b) preferably includes in addition one or more organic and/or inorganic acids.

One or more acids from the group comprising citric acid, tartaric acid, malic acid, lactic acid, acetic acid, sulfuric acid, hydrochloric acid, phosphoric acid, methanesulfonic acid, benzoic acid, malonic acid, oxalic acid, and/or 1-hydroxyethane-1,1-diphosphonic acid have proven to be suitable for adjusting the pH. The acid(s) is/are preferably selected from the group comprising citric acid, tartaric acid, malic acid, lactic acid, methanesulfonic acid, oxalic acid, malonic acid, benzoic acid, hydrochloric acid, sulfuric acid, phosphoric acid, and/or 1-hydroxyethane-1,1-diphosphonic acid.

Also likewise particularly preferred, therefore, is a multicomponent packaging unit (kit of parts), which is characterized in that
agent (b) in container (B) includes in addition
(b2) one or more acids from the group comprising citric acid, tartaric acid, malic acid, lactic acid, methanesulfonic acid, oxalic acid, malonic acid, benzoic acid, hydrochloric acid, sulfuric acid, phosphoric acid, and/or 1-hydroxyethane-1,1-diphosphonic acid.

Also likewise particularly preferred is a multicomponent packaging unit (kit of parts), which is characterized in that agent (b) in container (B) has
(b3) a pH of 1 to 6, preferably of 1.3 to 4.5, more preferably of 1.6 to 4.0, and particularly preferably of 2.0 to 3.6 (measured using a type N 61 glass electrode from the company Schott at a temperature of 22° C.).

Although the pH of agent (b) is preferably in the acidic range, the agents for finely adjusting the pH nevertheless can contain small amounts of alkalizing agents. The alkalinizing agents that can be used for this purpose according to the invention can be selected from the group formed by ammonia, alkanolamines, basic amino acids, and inorganic alkalinizing agents such as alkali (alkaline earth) metal hydroxides, alkali (alkaline earth) metal metasilicates, alkali (alkaline earth) metal phosphates, and alkali (alkaline earth) metal hydrogen phosphates. Suitable inorganic alkalinizing agents are sodium hydroxide, potassium hydroxide, sodium silicate, and sodium metasilicate. Organic alkalinizing agents that can be used according to the invention can be selected from monoethanolamine, 2-amino-2-methylpropanol, and triethanolamine. The basic amino acids that can be used as alkalinizing agents according to the invention can be selected from the group formed by arginine, lysine, ornithine, and histidine.

Agent (b) is provided as a liquid formulation to which further surface-active substances can be added. They are preferably selected from anionic, zwitterionic, amphoteric, and nonionic surfactants.

Agent (b) can contain as anionic surfactants, for example, fatty acids, alkyl sulfates, alkyl ether sulfates, and ether carboxylic acids having 10 to 20 C atoms in the alkyl group and up to 16 glycol ether groups in the molecule.

Agent (b) can also contain one or more zwitterionic surfactants such as, for example, betaine, N-alkyl-N,N-dimethylammonium glycinate, N-acylaminopropyl-N,N-dimethylammonium glycinate, and 2-alkyl-3-carboxymethyl-3-hydroxyethyl imidazoline.

Agents (b) suitable according to the invention are characterized further in that agent (b) includes in addition at least one amphoteric surfactant. Preferred amphoteric surfactants are N-alkylglycines, N-alkylpropionic acids, N-alkylaminobutyric acids, N-alkyliminodipropionic acids, N-hydroxyethyl-N-alkylamidopropyl glycines, N-alkyltaurines, N-alkylsarcosines, 2-alkylaminopropionic acids, and alkylaminoacetic acids. Particularly preferred amphoteric surfactants are N-cocoalkyl aminopropionate, cocoacylaminoethylamino propionate, and C₁₂-C₁₈ acylsarcosine.

It has proven especially advantageous if agent (b) includes in addition nonionic surfactants (b4). Preferred nonionic surfactants are
alkyl polyglycosides
alkylene oxide adducts to fatty alcohols and fatty acids with in each case 10 to 60 mol of ethylene oxide per mole of fatty alcohol or fatty acid, and
fatty acid triglycerides, which are ethoxylated with 10 to 60 ethylene oxide units.

Also likewise particularly preferred, therefore, is a multicomponent packaging unit (kit of parts), which is characterized in that agent (b) in container (B) includes in addition
(b4) one or more nonionic surfactants from the group of
   C₁₂-C₃₀ fatty alcohols, which are ethoxylated with 10 to 60 ethylene oxide units, and/or
   fatty acid triglycerides, which are ethoxylated with 10 to 60 ethylene oxide units.

The nonionic, zwitterionic, amphoteric and/or anionic surfactants can be used in amounts of 0.1 to 25.0% by weight, preferably 0.3 to 15.0% by weight, and very particularly preferably of 0.5 to 5.0% by weight, based on the total weight of agent (b).

Decolorization of Dyed Keratin Fibers

The multicomponent packaging unit of the invention is a system comprising agents (a) and (b) and is used for the decolorization of previously dyed keratinic fibers, in particular human hair. Dyed keratin fibers are typically fibers that were dyed previously with conventional oxidation dyes, known to the skilled artisan, and/or direct dyes.

The decolorizing agents are suitable for removing colors produced with oxidation dyes based on developer and coupler components on the keratin fibers. If the following compounds were used as developer, the colors produced with them can be removed readily, effectively, and almost without later redarkening by using the decolorizing agent: p-phenylenediamine, p-toluylenediamine-N,N-bis(β-hydroxyethyl)-p-phenylenediamine, 4-N,N-bis(β-hydroxyethyl)amino-2-methylaniline, 2-(β-hydroxyethyl)-p-phenylenediamine, 2-(α,β-dihydroxyethyl)-p-phenylenediamine, 2-hydroxymethyl-p-phenylenediamine, bis(2-hydroxy-5-aminophenyl)methane, p-aminophenol, 4-amino-3-methylphenol, 2,4,5,6-tetraaminopyrimidine, 4-hydroxy-2,5, 6-triaminopyrimidine, 2-hydroxy-4,5,6-triaminopyrimidine, and/or 4,5-diamino-1-(β-hydroxyethyl)pyrazole.

If the following compounds were used as coupler, the colors produced herewith can also be removed with a very good decolorizing result: m-phenylenediamine derivatives, naphthols, resorcinol and resorcinol derivatives, pyrazolones, and m-aminophenol derivatives. Suitable as coupler substances are in particular 1-naphthol, 1,5-, 2,7-, and 1,7-dihydroxynaphthalene, 5-amino-2-methylphenol, m-aminophenol, resorcinol, resorcinol monomethyl ether, m-phenylenediamine, 1-phenyl-3-methylpyrazolone-5,2,4-dichloro-3-aminophenol, 1,3-bis(2′,4′-diaminophenoxy)propane, 2-chlororesorcinol, 4-chlororesorcinol, 2-chloro-6-methyl-3-aminophenol, 2-amino-3-hydroxypyridine, 2-methylresorcinol, 5-methylresorcinol, and 2-methyl-4-chloro-5-aminophenol, 1-naphthol, 1,5-dihydroxynaphthalene, 2,7-dihydroxynaphthalene, 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcinol, and 2,6-dihydroxy-3,4-dimethylpyridine.

The substrate to be decolorized can also have been dyed with direct dyes. In this case, in particular nitrophenylenediamines, nitroaminophenols, azo dyes, anthraquinones, or indophenols may be used as direct dyes. Preferred direct dyes are the compounds known with the international names or trade names: HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52, and 1,4-diamino-2-nitrobenzene, 2-amino-4-nitrophenol, 1,4-bis(β-hydroxyethyl)-amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-1-hydroxy-4-nitrobenzene.

Furthermore, the substrates to be decolorized can also be dyed with naturally occurring or natural dyes, as they are included, for example, in henna red, henna neutral, henna black, chamomile blossom, sandalwood, black tea, buckthorn bark, sage, logwood, madder root, catechu, sedre, and alkanna root.

The decolorizing agents of the invention are intended for removing these colors and therefore themselves preferably contain no dyes, i.e., no oxidation dye precursors of the developer and coupler type and also no direct dyes.

In a further preferred embodiment, a multi-component packaging unit (kit of parts) of the invention is therefore characterized in that
the total amount of all dyes, included in agent (a), and oxidation dye precursors is a value of a maximum of 0.2% by weight, preferably of a maximum of 0.1% by weight, more preferably of a maximum of 0.05% by weight, and particularly preferably of a maximum of 0.01% by weight, based on the total weight of agent (a), and
the total amount of all dyes, included in agent (b), and oxidation dye precursors is a value of a maximum of 0.2% by weight, preferably of a maximum of 0.1% by weight, more preferably of a maximum of 0.05% by weight, and particularly preferably of a maximum of 0.01% by weight, based on the total weight of agent (b).

Oxidizing Agents

The multicomponent packaging unit of the invention is used for the reductive decolorization of dyed keratinic fibers. Agents (a) and (b) in this case together form the ready-to-use decolorizing agent, which includes a reducing agent. Agents (a) and (b) therefore preferably contain no oxidizing agents for reasons of incompatibility and to prevent exothermic, uncontrollable reactions.

Oxidizing agents in this case are understood to be in particular the oxidizing agents also used for the oxidative decolorization, such as, for example, hydrogen peroxide and persulfates (potassium persulfate (alternatively potassium peroxodisulfate), sodium persulfate (sodium peroxodisulfate), and ammonium persulfate (alternatively ammonium peroxodisulfate)). Preferably therefore none of the agents (a) and (b) contain the aforementioned oxidizing agents.

In a further preferred embodiment, a multi-component packaging unit (kit of parts) of the invention for this reason is characterized in that
the total amount of all oxidizing agents from the group of peroxides and persulfates, as included in agent (a), is a value of a maximum of 0.2% by weight, preferably of a maximum of 0.1% by weight, more preferably of a maximum of 0.05% by weight, and particularly preferably of a maximum of 0.01% by weight, based on the total weight of agent (a), and
the total amount of all oxidizing agents from the group of peroxides and persulfates, as included in agent (b), is a value of a maximum of 0.2% by weight, preferably of a maximum of 0.1% by weight, more preferably of a maximum of 0.05% by weight, and particularly preferably of a maximum of 0.01% by weight, based on the total weight of agent (b).

Other Ingredients

Further, agents (a) and (b) of the invention can contain other active substances, auxiliary substances, and additives, such as, for example, nonionic polymers such as, for example, vinylpyrrolidinone/vinyl acrylate copolymers, polyvinylpyrrolidinone, vinylpyrrolidinone/vinyl acetate copolymers, polyethylene glycols, and polysiloxanes; additional silicones such as volatile or nonvolatile, straight-chain, branched or cyclic, crosslinked or noncrosslinked polyalkylsiloxanes (such as dimethicones or cyclomethicones), polyarylsiloxanes, and/or polyalkylarylsiloxanes, particularly polysiloxanes with organofunctional groups, such as substituted or unsubstituted amines (amodimethicones), carboxyl, alkoxy, and/or hydroxyl groups (dimethicone copolyols), linear polysiloxanes(A)-polyoxyalkylene(B) block copolymers, grafted silicone polymers; cationic polymers such as quaternized cellulose ethers, polysiloxanes with quaternary groups, dimethyldiallylammonium chloride polymers, acrylamide-dimethyldiallylammonium chloride copolymers, dimethylaminoethylmethacrylate-vinylpyrrolidinone copolymers quaternized with diethylsulfate, vinylpyrrolidinone-imidazolinium-methochloride copolymers, and quaternized polyvinyl alcohol; zwitterionic and amphoteric polymers; anionic polymers such as, for example, polyacrylic acids or crosslinked polyacrylic acids; structurants such as glucose, maleic acid, and lactic acid, hair-conditioning compounds such as phospholipids, for example, lecithin and kephalins; perfume oils, dimethyl isosorbide, and cyclodextrins; fiber-structure-improving active substances, particularly mono-, di-, and oligosaccharides such as, for example, glucose, galactose, fructose, fruit sugar, and lactose; dyes for coloring the agent; antidandruff agents such as piroctone olamine, zinc omadine, and climbazole; amino acids and oligopeptides; protein hydrolysates with an animal and/or vegetable base, and in the form of their fatty acid condensation products or optionally anionically or cationically modified derivatives; vegetable oils; light stabilizers and UV blockers; active substances such as panthenol, pantothenic acid, pantolactone, allantoin, pyrrolidinone carboxylic acids, and salts thereof, as well as bisabolol; polyphenols, particularly hydroxycinnamic acids, 6,7-dihydroxycoumarins, hydroxybenzoic acids, catechins, tannins, leukoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetration agents such as glycerol, propylene glycol monoethyl ether, carbonates, hydrogen carbonates, guanidines, ureas, and primary, secondary, and tertiary phosphates; opacifiers such as latex, styrene/PVP and styrene/acrylamide copolymers; pearlescent agents such as ethylene glycol mono- and distearate and PEG-3 distearate; pigments and propellants such as propane-butane mixtures, N₂O, dimethyl ether, CO₂, and air. In this regard, reference is expressly made to known monographs, e.g., Kh. Schrader, Grundlagen und Rezepturen der Kosmetika [Fundamentals and Formulations of Cosmetics], 2^{nd} edition, Hüthig Buch Verlag, Heidelberg, 1989, which report the corresponding knowledge of the skilled artisan.

Mixing Ratio of Agents (a) and (b)

As already previously described, the ready-to-use decolorizing agent is prepared by mixing agents (a) and (b). In principle, agents (a) and (b) can be mixed in this case in different mixing ratios such as, for example, (a)/(b) of 20:1 to 1:20.

Agent (a) is preferably a solid, powdered, in particular preferably a paste-like agent. So that upon mixing with agent (b) the agent can also be completely transferred into solution, it is advantageous, however, to use agent (b) at least in the same amount as agent (a). It is preferred, furthermore, to use agent (b) in excess.

In a further preferred embodiment, a multicomponent packaging unit of the invention is therefore characterized in that the amounts of agent (a) in container (A) and of agent (b) in container (B) are selected so that when the application is prepared, i.e., during the mixing of agents (a) and (b), the mixing ratio (a)/(b) has a value of at most 1, preferably at most 0.9, more preferably at most 0.8, and particularly preferably at most 0.7.

To prepare the mixture, for example, agent (a) can be transferred from container (A) completely to container (B), which already includes agent (b). In this case, the size of container (B) is selected so that container (B) can accommodate the total amount of agents (a) and (b) and also allows mixing of both agents (a) and (b), e.g., by shaking or stirring.

By analogy, the preparation of the mixture can also occur by the complete transfer of agent (b) from container (B) to container (A), which already includes agent (a). In this case, the size of container (A) is to be selected so that container (A) can accommodate the total amount of agents (a) and (b) and also allows mixing of both agents (a) and (b), e.g., by shaking or stirring.

A further option for preparing the application mixture is the complete transfer of both agents (a) and (b) from containers (A) and (B) to a third container (C), which then allows the mixing of both agents, e.g., by shaking, or stirring.

Example: A multicomponent packaging unit of the invention includes
25 g of agent (a) in container (A)
100 g of agent (b) in container (B)

To prepare the application mixture, agent (b) is transferred completely from container (B) to container (A). Agents (a) and (b) are then shaken or stirred. The mixing ratio of agents (a)/(b) has a value of (25 g/100 g)=0.25.

Method

The previously described multicomponent packaging units (kit of parts) of the invention can be used in methods for the reductive decolorization.

A second subject of the present invention, therefore, is a method for the reductive decolorization of dyed keratinic fibers, comprising the following steps in the indicated sequence
(I) preparing a ready-to-use decolorizing agent by mixing an agent (a) with an agent (b), wherein agent (a) is an agent as was defined in the description of the first subject of the invention, and agent (b) is an agent as was defined in the description of the first subject of the invention,
(II) applying the ready-to-use decolorizing agent to dyed keratinic fibers,
(III) allowing the decolorizing agent to act for a time period of 5 to 60 minutes, preferably of 10 to 55 minutes, more preferably of 20 to 50 minutes, and particularly preferably of 30 to 45 minutes,
(IV) rinsing the decolorizing agent off the keratinic fibers,
(V) optionally applying an aftertreatment agent to the keratinic fibers, wherein the aftertreatment agent includes at least one amphoteric, zwitterionic, and/or anionic surfactant,
(VI) optionally rinsing the aftertreatment agent off the keratinic fibers.

Steps (I), (II), (III), and (IV) of the method represent the decolorizing process of the keratin fibers and are accordingly carried out in a direct time sequence one after another. There is basically no time limitation for the sequence of steps (IV) and (V). Thus, step (V) can occur for hours, days, or, for example, also up to two weeks after the end of step (IV).

As previously described, agents (a) and (b) are preferably used in a quantitative ratio (a)/(b), the value of which is at most 1, preferably at most 0.9, more preferably at most 0.8, and particularly preferably at most 0.7.

Accordingly, therefore, a method for decolorizing dyed keratinic fibers is also preferred, which is characterized in that the ready-to-use decolorizing agent is prepared in step (I) by mixing agent (a) with agent (b), wherein the two agents are used in a quantitative ratio (a)/(b), the value of which is at most 1, preferably at most 0.9, more preferably at most 0.8, and particularly preferably at most 0.7.

The aftertreatment agent optionally usable in process step (V) and (VI) can be, for example, a shampoo, a conditioner, a gel, or a solution.

An aftertreatment agent can be used in particular to prevent a redarkening or reoxidation, which can occur when atmospheric oxygen acts on decolorized keratin fibers. To prevent said reoxidation effectively, the aftertreatment should occur before atmospheric oxygen has time to act on the reduced keratin fibers. For this reason, the aftertreatment if possible should take place immediately after the decolorization (i.e., in time immediately after process step (IV) was completed). For this reason, it is preferable, if there is a time period of a maximum of 12 hours, preferably of a maximum of 6 hours, more preferably of a maximum of 1 hour, and particularly preferably of a maximum of 30 minutes between the completion of process step (IV) and the start of process step (V).

A preferred method of the invention therefore is characterized in that there is a time period of a maximum of 12 hours, preferably of a maximum of 6 hours, more preferably of a maximum of 1 hour, and particularly preferably of a maximum of 30 minutes between process steps (IV) and (V).

The use of the aftertreatment agent can also be repeated several times, for example, if the aftertreatment agent is a shampoo that is routinely used after the decolorization. If the aftertreatment, i.e., carrying out steps (V) to (VII), is repeated, it is therefore possible to suppress the reoxidation for an especially long time period.

Particularly preferred therefore is a method for the reductive decolorization of dyed keratinic fibers, comprising the following steps in the indicated sequence
(I) preparing a ready-to-use decolorizing agent by mixing an agent (a) with an agent (b), wherein agent (a) is an agent as was defined in the description of the first subject of the invention, and agent (b) is an agent as was defined in the description of the first subject of the invention
(II) applying the ready-to-use decolorizing agent to dyed keratinic fibers,
(III) allowing the decolorizing agent to act for a time period of 5 to 60 minutes, preferably of 10 to 55 minutes, more preferably of 20 to 50 minutes, and particularly preferably of 30 to 45 minutes,
(IV) rinsing the decolorizing agent off the keratinic fibers,
(V) applying an aftertreatment agent to the keratinic fibers, wherein the aftertreatment agent includes at least one amphoteric, zwitterionic, and/or anionic surfactant,
(VI) rinsing the aftertreatment agent off the keratinic fibers,
(VII) applying an aftertreatment agent to the keratinic fibers, wherein the aftertreatment agent includes at least one amphoteric, zwitterionic, and/or anionic surfactant,
(VIII) rinsing the aftertreatment agent off the keratinic fibers.

To prevent reoxidation or redarkening, the aftertreatment agent applied in process step (V) preferably includes in addition at least one acid from the group comprising citric acid, tartaric acid, malic acid, lactic acid, acetic acid, hydroxyethane-1,1-diphosphonic acid, methanesulfonic acid, benzoic acid, hydrochloric acid, sulfuric acid, phosphoric acid, malonic acid, and/or oxalic acid.

A particularly preferred method for the reductive decolorization of dyed keratinic fibers, therefore, is characterized further in that the aftertreatment agent applied in step (V) includes at least one acid from the group comprising citric acid, tartaric acid, malic acid, lactic acid, acetic acid, hydroxyethane-1,1-diphosphonic acid, methanesulfonic acid, benzoic acid, hydrochloric acid, sulfuric acid, phosphoric acid, malonic acid, and/or oxalic acid.

In other words, very particularly preferred therefore is a method for the reductive decolorization of dyed keratinic fibers, comprising the following steps in the indicated sequence
(I) preparing a ready-to-use decolorizing agent by mixing an agent (a) with an agent (b), wherein agent (a) is an agent as was defined in the description of the first subject of the invention, and agent (b) is an agent as was defined in the description of the first subject of the invention,
(II) applying the ready-to-use decolorizing agent to dyed keratinic fibers,
(III) allowing the decolorizing agent to act for a time period of 5 to 60 minutes, preferably of 10 to 55 minutes, more preferably of 20 to 50 minutes, and particularly preferably of 30 to 45 minutes,
(IV) rinsing the decolorizing agent off the keratinic fibers,
(V) applying an aftertreatment agent to the keratinic fibers, wherein the aftertreatment agent
   includes at least one amphoteric, zwitterionic, and/or anionic surfactant, and
   includes at least one acid from the group comprising citric acid, tartaric acid, malic acid, lactic acid, acetic acid, hydroxyethane-1,1-diphosphonic acid, methanesulfonic acid, benzoic acid, hydrochloric acid, sulfuric acid, phosphoric acid, malonic acid, and/or oxalic acid, and
(VI) optionally rinsing the aftertreatment agent off the keratinic fibers.

The statements made regarding the agent of the invention apply mutatis mutandis with respect to other preferred embodiments of the method of the invention.

### Examples

1.1. Coloring

The following formulations were prepared (all quantities are given in % by weight):

### Color Cream (F1)

| | % by |
|---|---|
| Raw material | weight |
| Cetearyl alcohol | 8.5 |
| C12-C18 fatty alcohols | 3.0 |
| Ceteareth-20 | 0.5 |
| Ceteareth-12 | 0.5 |
| Plantacare 1200 UP (lauryl glucoside, 50-53% aqueous solution) | 2.0 |
| Sodium laureth-6 carboxylate (21% aqueous solution) | 10.0 |
| Sodium myreth sulfate (68-73% aqueous solution) | 2.8 |
| Sodium acrylate, trimethylammoniopropylacrylamide chloride | 3.8 |
| copolymer (19-21% aqueous solution) | |
| Potassium hydroxide | 0.83 |
| p-Toluylenediamine, sulfate | 2.25 |
| m-Aminophenol | 0.075 |
| 2-Amino-3-hydroxypyridine | 0.12 |
| Resorcinol | 0.62 |
| 4-Chlororesorcinol | 0.26 |
| 3-Amino-2-methylamino-6-methoxypyridine | 0.04 |
| 1,3-Bis(2,4-diaminophenoxy)propane, tetrahydrochloride | 0.05 |
| Ammonium sulfate | 0.1 |
| Sodium sulfite | 0.4 |
| Ascorbic acid | 0.1 |
| 1-Hydroxyethane-1,1-diphosphonic acid (60% aqueous solution) | 0.2 |
| Ammonia (25% aqueous solution) | 7.2 |
| Water | To 100 |

Oxidizing Agents (Ox)

| | % by |
|---|---|
| Raw material | weight |
| Sodium benzoate | 0.04 |
| Dipicolinic acid | 0.1 |
| Disodium pyrophosphate | 0.1 |
| Potassium hydroxide | 0.09 |
| 1,2-Propylene glycol | 1.0 |
| 1-Hydroxyethane-1,1-diphosphonic acid (60% aqueous solution) | 0.25 |
| Liquid paraffin | 0.30 |
| Steartrimonium chloride | 0.39 |
| Cetearyl alcohol | 3.4 |
| Ceteareth-20 | 1.0 |
| Hydrogen peroxide (50% aqueous solution) | 12.0 |

The color cream (F1) and the oxidizing agent (Ox) were mixed in a 1:1 quantitative ratio and applied to hair strands (Kerling Euronaturhaar [Euro natural hair] white). The application mixture:hair weight ratio was 4:1, and the contact time was 30 minutes at a temperature of 32° C. The strands were then rinsed with water, dried, and allowed to rest for at least 24 hours at room temperature. The strands were dyed using a dark-brown shade.

1.2. Decolorization

The following decolorizing agents were prepared (all quantities are given in % by weight of active substance):

Agent (a)

| Agent (a) | Agent (a1) | Agent (a2) |
|---|---|---|
| Versagel M 1600 ⁽¹⁾ | 3.75 | 6.0 |
| Lanette N ⁽²⁾ | 5.25 | 8.4 |
| Ceteareth-20 (C16-C18 fatty alcohol, | 0.37 | 0.6 |
| ethoxylated with 20 EO) | | |
| Ceteareth-50 (C16-C18 fatty alcohol, | 2.25 | 3.6 |
| ethoxylated with 50 EO) | | |
| Sodium dithionite | 44.0 | 17.6 |
| Liquid paraffin | to 100 | to 100 |

⁽¹⁾ INCI: Paraffinium Liquidum (Mineral Oil), Ethylene/Propylene/Styrene Copolymer, Butylene/Ethylene/Styrene Copolymer
⁽²⁾ INCI: Cetearyl alcohol (ca. 90%) and Sodium Cetearyl Sulfate (ca. 10.0%)

Agent (b)

| Agent (b) | Agent (b1) |
|---|---|
| Lanette N ⁽²⁾ | 3.8 |
| PEG-40 Castor Oil | 0.7 |
| 1-Hydroxyethane-1,1-diphosphonic acid | 0.5 |
| (60%) | |
| Water | to 100 |

Agents (a1) and (b1) were stirred together in the quantitative ratio (a1)/(b1) of 1:4 (i.e., (a1)/(b1)=0.25). Agents (a1) and (b1) can be mixed together without any dust formation; a homogeneous emulsion could be obtained even after 1 minute of stirring.

The ready-to-use decolorizing agent prepared in this way was applied to hair colored under Section 1.1 and left to act for 45 minutes at a temperature of 30° C. The strands were then rinsed with water for 20 seconds. The hair was almost completely decolorized with a very uniform result.

Agents (a2) and (b1) were stirred together in the quantitative ration (a2)/(b1) of 1:1 (i.e., (a2)/(b1)=1). Agents (a2) and (b1) can be mixed together without any dust formation; a homogeneous emulsion could be obtained even after 1 minute of stirring.

The ready-to-use decolorizing agent prepared in this way was applied to hair colored under Section 1.1 and left to act for 45 minutes at a temperature of 30° C. The strands were then rinsed with water for 20 seconds. The hair was almost completely decolorized with a very uniform result.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A multicomponent packaging unit (kit of parts) for the reductive decolorization of dyed keratinic fibers, comprising, separately formulated from one another
(I) a container (A) containing a cosmetic agent (a) and
(II) a container (B) containing a cosmetic agent (b),
wherein
agent (a) in container (A) includes
(a1) one or more reducing agent selected from the group consisting of: sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, sulfanylacetic acid (thioglycolic acid), and/or ascorbic acid, and
(a2) one or more fatty components from the group comprising C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, C₁₂-C₃₀ fatty acid esters, hydrocarbons, and/or silicone oils,
and has
(a3) a water content of at most 10.0% by weight, based on the total weight of agent (a), and
agent (b) in container (B) has
(b1) a water content of at most 30.0% by weight, based on the total weight of agent (b).

2. The multicomponent packaging unit (kit of parts) according to claim 1, wherein agent (a) in container (A) includes
(a1) one or more reducing agents selected from the group comprising sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate and/or ammonium thiosulfate in a total amount of 10.0 to 90.0% by weight.

3. The multicomponent packaging unit (kit of parts) according to claim 1, wherein agent (a) in container (A) includes the fatty component(s) from group (a2) in a total amount of 10.0 to 90.0% by weight based on the total weight of agent (a).

4. The multicomponent packaging unit (kit of parts) according to claim 1, wherein agent (a) in container (A) includes the fatty component(s) from group (a2) in a total amount of 30.0 to 80.0% by weight based on the total weight of agent (a).

5. The multicomponent packaging unit (kit of parts) according to claim 1, wherein agent (a) in container (A) includes (a2) one or more hydrocarbons in a total amount of 15.0 to 90.0% by weight based on the total weight of agent (a).

6. The multicomponent packaging unit (kit of parts) according to claim 1, wherein agent (a) in container (A) includes (a2) one or more hydrocarbons in a total amount of 30.0 to 75.0% by weight based on the total weight of agent (a).

7. The multicomponent packaging unit (kit of parts) according to claim 1, wherein agent (a) in container (A) has (a3) a water content of at most 8.0% by weight based on the total weight of agent (a).

8. The multicomponent packaging unit (kit of parts) according to claim 1, wherein agent (a) in container (A) has (a3) a water content of at most 1.0% by weight based on the total weight of agent (a).

9. The multicomponent packaging unit (kit of parts) according to claim 1, wherein agent (a) in container (A) includes in addition (a4) one or more nonionic surfactants in a total amount of 0.1 to 15.0% by weight based on the total weight of agent (a).

10. The multicomponent packaging unit (kit of parts) according to claim 1, wherein agent (a) in container (A) further comprises (a5) one or more nonionic polymers in a total amount of 0.1 to 15.0% by weight based on the total weight of agent (a).

11. The multicomponent packaging unit (kit of parts) according to claim 1, wherein agent (b) in container (B) has (b1) a water content of at least 40.0% by weight based on the total weight of agent (b).

12. The multicomponent packaging unit (kit of parts) according to claim 1, wherein agent (b) in container (B) further comprises (b2) one or more acids selected from the group consisting of citric acid, tartaric acid, malic acid, lactic acid, methanesulfonic acid, oxalic acid, malonic acid, benzoic acid, hydrochloric acid, sulfuric acid, phosphoric acid, and 1-hydroxyethane-1,1-diphosphonic acid.

13. The multicomponent packaging unit (kit of parts) according to claim 1, wherein agent (b) in container (B) has (b3) a pH of 1 to 6.

14. The multicomponent packaging unit (kit of parts) according to claim 1, wherein agent (b) in container (B) has (b3) a pH of 2.0 to 3.6.

15. The multicomponent packaging unit (kit of parts) according to claim 1, wherein agent (b) in container (B) further comprises (b4) one or more nonionic surfactants selected from the group consisting of: C₁₂-C₃₀ fatty alcohols, which are ethoxylated with 10 to 60 ethylene oxide units, and fatty acid triglycerides, which are ethoxylated with 10 to 60 ethylene oxide units.

16. The multicomponent packaging unit (kit of parts) according to claim 1, wherein the total amount of all dyes, included in agent (a), and oxidation dye precursors is a value of a maximum of 0.2% by weight, and the total amount of all dyes in agent (b), and oxidation dye precursors 0.2% by weight or less based on the total weight of agent (b).

17. The multicomponent packaging unit (kit of parts) according to claim 1, wherein the total amount of all oxidizing agents from the group of peroxides and persulfates, as included in agent (a), is a value of a maximum of 0.2% by weight based on the total weight of agent (a), and the total amount all oxidizing agent from group of peroxides and persulfates as included in agent (b), is a value of maximum of 0.2% by weigh based on the total weight of agent (b).

18. A method for the reductive decolorization of dyed keratinic fibers, comprising the following steps in the indicated sequence
(I) preparing a ready-to-use decolorizing agent by mixing an agent (a) with an agent (b), wherein
agent (a) is an agent as was defined in claim 1,
agent (b) is an agent as was defined in claim 1,
(II) applying the ready-to-use decolorizing agent to dyed keratinic fibers,
(III) allowing the decolorizing agent to act for a time period of 5 to 60 minutes,
(IV) rinsing the decolorizing agent off the keratinic fibers,
(V) optionally applying an aftertreatment agent to the keratinic fibers, wherein the aftertreatment agent includes at least one amphoteric, zwitterionic, and/or anionic surfactant,
(VI) optionally rinsing the aftertreatment agent off the keratinic fibers.

19. The method according to claim 18, characterized in that the aftertreatment agent, applied in step (V), includes at least one acid selected from the group consisting of citric acid, tartaric acid, malic acid, lactic acid, acetic acid, hydroxyethane-1,1-diphosphonic acid, methanesulfonic acid, benzoic acid, hydrochloric acid, sulfuric acid, phosphoric acid, malonic acid, and oxalic acid.
